(19) United States
(12) Patent Application Publication
(10) Pub. No.: US 2017/0076414 A1
(43) Pub. Date: Mar. 16, 2017

(54) APPARATUSES AND METHODS FOR AUTO-REPLENISHMENT OF ELECTRODES FOR TRANSDERMAL ELECTRICAL STIMULATION

(71) Applicants: Jason EGNAL, Menlo Park, CA (US); Daniel Z. WETMORE, Brooklyn, NY (US)

(72) Inventors: Jason EGNAL, Menlo Park, CA (US); Daniel Z. WETMORE, Brooklyn, NY (US)

(21) Appl. No.: 15/265,633

(22) Filed: Sep. 14, 2016

Related U.S. Application Data
(60) Provisional application No. 62/218,560, filed on Sep. 14, 2015.

(51) Int. Cl. G06Q 50/22; A61N 1/08; G06Q 30/06; A61N 1/36

(52) U.S. Cl. CPC G06Q 50/22; A61N 1/36014; A61N 1/08; G06Q 30/0635; A61N 2001/083

(57) ABSTRACT

Described herein are methods for automatically reordering, resupplying, or replenishing TES electrode apparatus units (“electrodes”) for use with a wearable neurostimulator.

### CROSS REFERENCE TO RELATED APPLICATIONS

This patent application claims priority to U.S. Provisional Patent Application No. 62/218,560, titled “SYSTEMS AND METHODS FOR AUTO-REPLENISHMENT OF TRANSDERMAL ELECTRICAL STIMULATION ELECTRODE APPARATUS UNITS,” and filed on Sep. 14, 2015.

This patent application may also be related to the following U.S. patent applications, which are herein incorporated by reference in their entirety: U.S. patent application Ser. No. 15/210,742, titled “METHODS FOR USER CONTROL OF NEUROSTIMULATION TO MODIFY A COGNITIVE STATE” and filed on Jul. 14, 2016; U.S. patent application Ser. No. 14/956,193, titled “TRANSDERMAL ELECTRICAL STIMULATION METHODS FOR MODIFYING OR INDUCING COGNITIVE STATE” and filed on Dec. 1, 2015;

U.S. patent application Ser. No. 14/091,121, titled “WEARABLE TRANSDERMAL ELECTRICAL STIMULATION DEVICES AND METHODS OF USING THEM” and filed on Nov. 26, 2013; and U.S. patent application Ser. No. 15/264,224, titled “APPARATUSES AND METHODS FOR NEUROMODULATION” and filed on Sep. 13, 2016; each of these is hereby incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are methods and systems for automatically resupplying transdermal electrical stimulation electrode apparatus units to a user (e.g. to be used for neuromodulation) on an as-needed basis.

### BACKGROUND

The brain is composed of neurons and other cell types in connected networks that process sensory inputs, generate motor commands, and control all other behavioral and cognitive functions. Neurons communicate primarily through electrochemical pulses that transmit signals between connected cells within and between brain areas. Noninvasive neuromodulation technologies that affect neuronal activity can modulate the pattern of neural activity and cause altered behavior, cognitive states, perception, and motor output without requiring an invasive procedure.

Transcranial/transdermal electric stimulation (hereinafter “TES”) through scalp electrodes has been used to affect brain function in humans. TES has been shown to improve motor control and motor learning, improve memory consolidation during slow-wave sleep, regulate decision-making and risk assessment, affect sensory perception, and cause movements. TES has been used therapeutically in various clinical applications, including treatment of pain, depression, epilepsy, and tinnitus. Despite the research to date on TES neurostimulation, existing methods and apparatuses for TES are lacking for applications related to sleep.

Consumable electrodes for TES improve the comfort and efficacy of TES neuromodulation when they are replaced at appropriate times. In order to do so, a user must have sufficient supply of new electrode apparatus units. However, consumers may find it difficult and bothersome to track their usage and re-order the consumable/disposable portions of the wearable neurostimulator. One simple solution is automatically reordering based on the amount of time elapsed between the user's last order/reorder; however this may lead to either periods during which the user may not have any electrodes (if the rate of automatic reordering is not sufficiently frequent) or periods during which the user has too many electrodes (if the rate of automatic reordering is too frequent). The problem is particularly acute in the context of a consumable portion of a neurostimulator, for which it may be difficult for the user to themselves determine the rate of consumption, particularly for electrodes, which may vary in their efficacy based on the type of usage they experience and the type/nature of the electrode(s).

Described herein are methods for automatically sending additional electrode apparatus units to a user to ensure they have sufficient supply to conduct TES sessions according to their usual pattern of TES usage. In general, described herein are methods of automatically replenishing a user's supply of electrode apparatus units for TES.

### SUMMARY OF THE DISCLOSURE

Described herein are methods for automatically reordering transdermal electrical stimulation (TES) electrode units (or other consumable/disposable components) of a wearable neurostimulator. For example, described herein are methods for estimating from the wearable neurostimulator apparatus or a hand-held electronic device, such as a smartphone, the count of TES electrode apparatus units that a user may have or is likely to need, and automatically ordering (reordering) these TES electrodes apparatus units.

For example, described herein are methods of automatically supplying an electrode apparatus for transdermal electrical stimulation (TES) to a user, the method comprising: storing an initial estimate of TES electrode apparatus units associated with a wearable neurostimulator apparatus in the wearable neurostimulator apparatus or a handheld processer in communication with the wearable neurostimulator apparatus; detecting use of a TES electrode apparatus unit and updating the initial estimate to a current estimate in the wearable neurostimulator apparatus or a handheld processer in communication with the wearable neurostimulator apparatus; comparing the current estimate to a threshold value and shipping, delivering, or otherwise supplying one or more new TES electrode apparatus units to the user if the current estimate falls below the threshold value; and updating the current estimate to include the one or more new TES electrode apparatus units.

A method of supplying an electrode apparatus for transdermal electrical stimulation (TES) to a user may include: storing an estimate of the number of electrode apparatus units remaining for the user; detecting a use of a TES electrode apparatus; updating the estimate of the number of electrode apparatus units remaining for the user; comparing the updated estimate to a threshold value; shipping, delivering, or otherwise supplying one or more new electrode apparatus units to the user; and updating the estimate of the number of electrode apparatus units remaining for the user to reflect the new received units.

In general, the current estimate may comprise a fractional number to reflect a partially used TES electrode apparatus unit. In some variations the method may include rounding up/down to a whole number of units. In some variations the method may include determining the rate of consumption of one or more TES electrode apparatus units; this rate may be used to determine the estimate of TES electrode apparatus units associated with a wearable neurostimulator apparatus. In some variations the method or an apparatus performing them may determine from the detected use of TES electrode apparatus units a rate of consumption of the TES electrode apparatus units and may trigger reordering based on the rate of use and consumption rate.

In any of these apparatuses described herein, the methods may include detecting use of the TES electrode apparatus unit by detecting use by the wearable neurostimulator apparatus. Use may be detected, for example, when the TES electrode apparatus unit is coupled to the wearable neurostimulator apparatus. Alternatively or additionally, detecting use may comprise identifying a specific TES electrode apparatus unit by one or more of: RFID, NFC, optical code sensing, QR code detection, identification chip detection. Identification may be identification of a specific individual TES electrode apparatus unit, or it may be detection of a type, class, category, family, lot, etc. of TES electrode apparatus unit; the method or apparatus may then use this identification number to determine characteristics, including typical shelf-life, rate of consumption, etc. for the TES electrode apparatus unit and may use this information to estimate consumption and need for additional TES electrode apparatus units. For example, detecting use of the specific TES electrode apparatus unit may comprise determining a remaining functional lifetime of the TES electrode apparatus unit based on a comparison of a manufacturing specification and a stimulation parameter. Any stimulation parameter may be used; for example the stimulation parameter may be total net charge transfer and/or the duration of electrical stimulation.

In any of these methods and apparatuses, detecting use may comprise indirectly detecting use based on activity of the wearable neurostimulator apparatus (wherein the TES electrode apparatus unit is coupled to the wearable neurostimulator apparatus). For example, detecting use may comprise correlating a physiological measurement with activity of the wearable neurostimulator apparatus. Detecting use may comprise measuring activity of the wearable TES electrode using an accelerometer and/or gyroscope associated with the wearable neurostimulator apparatus. In some variations, detecting use may comprise analyzing online activity of the user. Detecting use may comprise analyzing a semantic or other text or voice analysis of the user.

As mentioned, any of these methods (and apparatuses configured to perform them) may be configured to store the current estimate of TES electrode apparatus units in a machine-readable memory of the wearable neurostimulator apparatus. Such a machine readable memory may include non-transient computer readable media, such as software, firmware or the like. In some variations a register or memory may be used.

In general, any of these methods may include storing the current estimate of TES electrode apparatus units in a smartphone or other personal computing device. Typically this may be in a smartphone that is connected (including wirelessly connected, e.g. by Bluetooth or other wireless methods, and/or by a wired connection) to the wearable neurostimulator. The same controller that controls stimulation (e.g., application of TES waveforms) by the wearable neurostimulator may be used to perform any of the steps described herein, including estimating and detecting use of the TES electrode apparatus units.

In general, the estimate of TES electrode apparatus units remaining for the user may be transmitted via the Internet and stored in a remote server; the remote server may communicate with the user, and/or may automatically reorder and/or send additional TES electrode apparatus units.

The threshold value may be set by the user or automatically, based on user behavior and/or preferences. For example, any of these methods may include determining the threshold value for sending one or more new electrode apparatus units based on a pattern of the user's use of the wearable neurostimulator apparatus. Any of these methods may include periodically updating the threshold value based on the user's pattern of use of the wearable neurostimulator apparatus.

The number (count) of TES electrode apparatus units sent for a reorder may be estimated automatically and/or based on user preference. For example, the number of TES electrode apparatus units reordered upon reaching the threshold value may be selected by the user. Alternatively or additional, the number may be automatically selected (e.g., determined based on a pattern of use of the wearable neurostimulator apparatus).

The method of claim 20, wherein the number of electrode apparatus units supplied to a user upon reaching the threshold value is updated periodically based on a pattern of use or delay in use of the wearable neurostimulator apparatus.

Shipping, delivering, or otherwise supplying one or more new TES electrode apparatus units to the user may be achieved by any appropriate method, including, for example, a local courier or delivery service, fulfillment from a regional or national warehouse, etc. The speed of shipping, delivering, or otherwise supplying one or more new electrode apparatus units to the user may be performed based on a pattern of use of the wearable neurostimulator apparatus by the user, including the rate of usage.

In general, updating the current estimate may occur at a fixed time after shipping based on the shipment method and speed, and/or once a tracking, delivery signature, or other delivery notice is received. In some variations, updating the current estimate occurs when a first electrode apparatus of the shipment is detected by the wearable neurostimulator apparatus or a handheld processer in communication with the wearable neurostimulator apparatus. For any of these methods, the user may select from a plurality of choices for shipping.

Also described herein are apparatuses, including systems and devices, that may perform any of the methods described herein. For example, an apparatus may include a wearable neurostimulator device and/or a software/firmware or hardware portion of a handheld electronic device, such as a wearable electronic device (e.g., smartphone, pad, smart watch, etc.) that includes a register or memory storing the (initial and/or ongoing) estimate of TES electrode apparatus units associated with the wearable neurostimulator. The apparatus may also include one or more sensors for determining when the TES electrode apparatus unit is used (or partially used) and/or for determining the identity of the TES electrode apparatus unit, and/or for determining when the TES electrode apparatus unit is attached to the wearable neurostimulator apparatus. Any of these apparatuses may also include a processor, and/or one or more inputs configured to allow user input, as well as communications subsystems for communicating to a remote server to order or reorder, ship, deliver or otherwise supply the user associated with the wearable neurostimulator the additional TES electrode apparatus units.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A shows one example of a wearable neurostimulator apparatus (e.g., electrical stimulation apparatus) seen from a back view, that may be worn on a subject with an exemplary TES electrode apparatus unit.

FIG. 1B shows the wearable neurostimulator apparatus of FIG. 1A worn on a subject's head.

FIG. 2A shows one example of a TES electrode apparatus unit.

FIG. 2B shows another example of a TES electrode apparatus unit.

FIG. 3 shows a workflow for methods of automatically replenishing a user's supply of electrode apparatus units for TES.

### DETAILED DESCRIPTION

Described herein are methods for automatically reordering, resupplying, or replenishing TES electrode apparatus units (“electrodes”) for use with a wearable neurostimulator that may be controlled and worn by the user. A TES electrode apparatus unit may be a consumable portion of the wearable neurostimulator and may be used once or more than once (e.g. may have fractional use). In particular, described herein are methods and apparatuses for conveniently tracking and automatically ordering (or requesting/prompting a user to reorder) consumables such as TES electrode apparatus units based on the actual user activity and/or properties of the particular consumable (e.g., TES electrode apparatus units) used, and/or the user preferences.

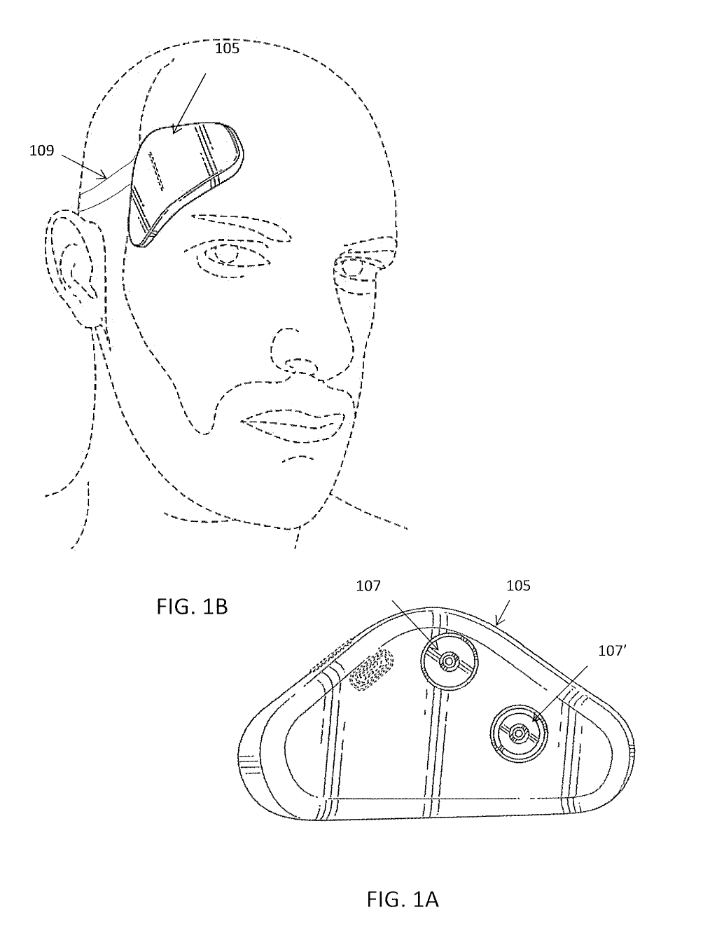

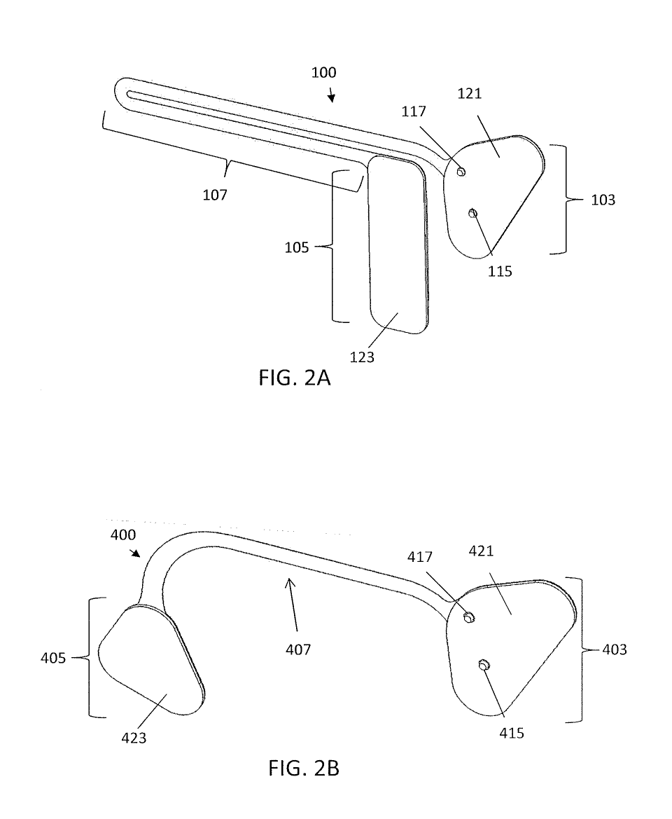

The methods and apparatuses described herein may be performed by any wearable neurostimulator, but particularly those that are self-contained, and user operated. For example, FIG. 1A illustrates a wearable neurostimulator that is connectable to a subject via attachment with a TES electrode apparatus unit (examples of which are shown in FIGS. 2A and 2B). The wearable neurostimulator may worn on any portion of the body, including, but not limited to, the head, neck, (head and neck), torso, arms, shoulder, back, chest, belly, leg, waist, crotch, feet, hands, or any combination thereof.

For example the apparatus shown in FIG. 1A includes a wearable housing that both encloses much of the electronics (e.g., a controller, current source, etc.) and also has a user-facing surface that is curved based on the region of the head where it will be positioned. In the example provided in FIG. 1A, the neurostimulator, to which the TES electrode apparatus unit attaches, may include mating junctions (openings, receptacles, female receivers, etc.) to receive and make electrical and mechanical contact with the connectors on the TES electrode apparatus unit. The wearable transdermal electrical neurostimulator may include a button for control by the subject. The subject may use the button to stop or reset the neurostimulator when necessary. In some embodiments, the wearable transdermal electrical stimulator may include a light indicator configured to provide visual feedback or a transducer for providing tactile feedback. For example, the light indicator can be used to indicate the connection of the electrodes, the status and progress of the TES stimulation session. For example, the tactile indicator can be used to indicate function of the device (e.g. 1 minute left in waveform; batteries low; placement incorrect, etc.) while worn on the head when a user cannot easily view a visual indicator without a mirror or front-facing camera of a smartphone or the like.

The wearable neurostimulator may include a housing 105 containing current control circuitry, fuse and other safety circuitry, wireless antenna and chipset, waveform generator, memory, microprocessor, and connectors to connect to the TES electrode apparatus unit (in FIG. 1A, two electrode connectors are shown 107, 107′). The housing may also contain a battery, recharging circuitry, and other electrical components. The housing may also include (or house) identification circuitry for identifying (automatically identifying) the TES electrode unit, including capacitive or other electrical identification circuitry (e.g., for identifying based on the electrical properties of a connected TES electrode apparatus unit), optical (camera, etc.) identification components, etc.

FIG. 1B illustrates a user wearing the wearable neurostimulator 105 on his temple; the neurostimulator is coupled to a wearable TES electrode apparatus unit 109 that connects adhesively to both the temple and the neck, and includes one or more connectors to connect to the wearable device. In some variations the TES electrode apparatus unit is configured to connect to the back of a user's neck. In some variations more than one TES electrode apparatus unit may couple to the same wearable neurostimulator; any of the methods described herein may therefore track the separate TES electrode apparatus units that are used for each operation of the device (and may be consumed at different rates).

FIGS. 2A and 2B illustrate examples of TES electrode apparatus units. In FIG. 2A, a front perspective view of a TES electrode apparatus unit is shown. The front side is the side that will face away from the subject when worn. This example of a TES electrode apparatus unit is thin, so that the electrode portions include a front side and a back side. The TES electrode apparatus unit in this example has a thin body that includes the electrode portions 103, 105 as well as an elongate body region 107 extending between the two electrode portions. The elongate body is also thin (having a much larger diameter and height than thickness).

In this example, two connectors 115, 117 (electrical and mechanical connectors, shown in this example as snaps) extend from the front of the cantilevered electrode apparatus. The front of the first electrical portion 103 may also include an optional foam and/or adhesive material 121 through which the snaps extend proud of the first electrical portion. The first electrical portion is shaped and sized so that the snaps will connect to plugs (ports, holders, opening, female mating, etc.) on the electrical stimulator. The connectors on the electrode apparatus may be spaced in the same manner as the complimentary connectors on the housing of the neurostimulator apparatus. The second electrode portion may also include an optional foam or backing portion 123. An active region of the electrodes may include a conductive material (e.g., electrically conductive gel). Similarly, the back of the second electrode portion 105 may include a second active region which is bounded on an upper and lower side by an adhesive. The adhesive may be any biocompatible adhesive that can releasably hold the material to the skin. The use of the TES electrode apparatus unit may include determining the consumption (fractional or whole-number consumption) or all or any portion of the TES electrode apparatus unit, such as the adhesive, the conductive material, etc. Either the wearable neurostimulator or an associated hand-held electronic device (which may be a dedicated device or it may be a generic device, such as a smartphone) may track the consumption (use) of the TES electrode apparatus unit and/or part of the TES electrode apparatus unit; one or more sensors for determining/detecting consumption may be used. In some variations the apparatus (wearable neurostimulator and/or handheld) may be configured to determine one or more electrical properties, such as skin impedance, of the TES electrode apparatus unit and estimate consumption/use based on this estimate.

FIG. 2B shows another, similar example of a TES electrode apparatus unit. This example is similar to the variation shown in FIG. 2A. The connectors (snaps 417, 415) are in the same position, as are the shape of the first electrode portion 403 and foam/backing material 421 (which may also or alternatively be an adhesive material) for connection to the back of the neurostimulator such as the one shown in FIG. 1A. This example may be used to fit to a user's head and/or neck. In particular, the portion of the substrate forming the elongate body region 407 extending between the two electrode portions 403, 405 is shaped slightly differently. In this example, the cantilevered electrode apparatus may be configured to connect, for example, to the subject's temple with the first electrode portion (to which the neurostimulator may be connected) and the elongate body region may be bent around the subject's head so that the second electrode portion may be in electrical contact with a region behind the subject's ear (e.g., at or near the mastoid). By placing the first active region of the first electrode portion 405 in electrical contact with the skin at the temple region, using the adhesive material surrounding the electrically active region to hold the electrically active region (and the attached neurostimulator) in position, the second electrically active region may also be adhesively held to skin so that the second electrically active region is in contact with the mastoid region.

In general, a wearable neurostimulator may include any of the features described herein. In general, these apparatuses may also be configured to be paired with and/or communicate with a handheld electronic device (e.g., smartphone, wearable electronics, etc.) that may modulate or control operation of the wearable neurostimulator, including tracking use and condition of the TES electrode apparatus units.

In general, a method of automatically supplying an electrode apparatus for transdermal electrical stimulation (TES) to a user may be performed at the level of the wearable electronics and/or at a handheld apparatus that interfaces with the wearable electronics (as used herein a handheld apparatus include a wearable device such as a smart watch, smart garment, smart jewelry, etc. that includes one or more processors and communicates with the neurostimulator and/or a remote server). For example, either the wearable neurostimulator and/or a handheld electronic device may be configured to store an initial estimate of TES electrode apparatus units associated with a wearable neurostimulator apparatus in the wearable neurostimulator apparatus or a handheld processer in communication with the wearable neurostimulator apparatus. The initial value may be manually or automatically set. For example, the initial value may be set to the number of TES electrode apparatus units shipped with the apparatus. Alternatively or additionally, the user may input manually the number of TES electrode apparatus unit initially.

Thereafter (and on an ongoing, continuous or periodic basis) the wearable neurostimulator or a processor in communication therewith may detect use of a TES electrode apparatus unit and update the initial estimate to a current estimate of TES electrode apparatus units associated with the wearable neurostimulator apparatus, in the wearable neurostimulator apparatus or a handheld processor in communication with the wearable neurostimulator apparatus. As discussed above, fraction use may be tracked as well, for one or any number of TES electrode apparatus unit coupled to the wearable neurostimulator. In some variations the wearable neurostimulator and/or a processor in communication therewith may determine consumption (use) of the TES electrode apparatus unit(s) at the end of a TES ‘session’ and/or during the session.

The wearable neurostimulator and/or a processor in communication therewith may also periodically (e.g., in an ongoing, continuous or periodic basis) compare the current estimate to a threshold value and shipping, delivering, or otherwise supplying one or more new TES electrode apparatus units to the user if the current estimate falls below the threshold value, and may update the current estimate to include the one or more new TES electrode apparatus units.

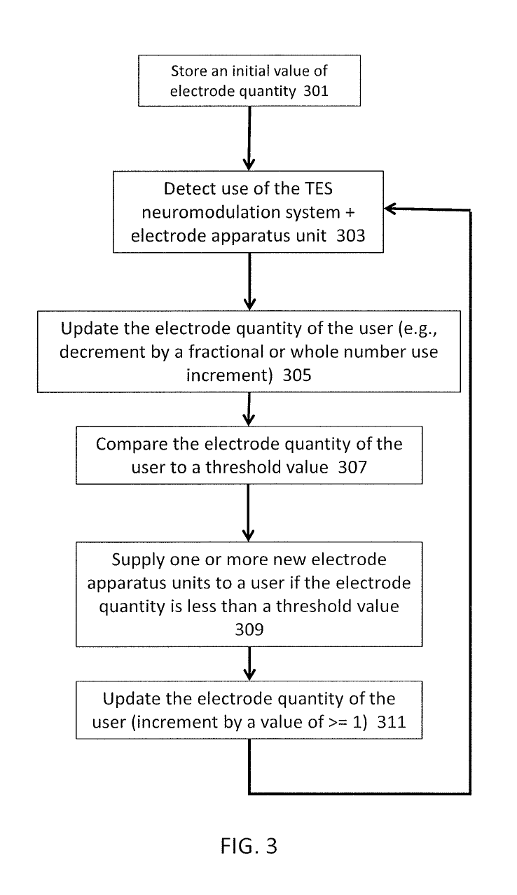

FIG. 3 outlines one example of such a method. For example, in FIG. 3, the wearable neurostimulator and/or a processor in communication therewith stores the initial number of the TES electrode apparatus units associated with a particular use (or alternatively a particular neurostimulator apparatus) 301. The wearable neurostimulator and/or a processor in communication therewith then detects the use of the TES electrode apparatus unit(s) before, during or after operation of the wearable neurostimulator 303, and may then update (decrement) the current number (value) of TES electrode apparatus units 305. Periodically (or alternatively continuously) the apparatus may compare the current number (value) to a threshold value for the number of TES electrode apparatus units 307, and if that number is below a threshold, may automatically reorder TES electrode apparatus units 309. Once the order has been made, shipped, and/or received, the apparatus may adjust the current number to include these newly ordered/received electrodes 311.

As mentioned above, any of these methods may be performed automatically by an apparatus, such as a wearable neurostimulator and/or a processor associated therewith (e.g., a hand-held device such as a smartphone).

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of automatically supplying an electrode apparatus for transdermal electrical stimulation (TES) to a user, the method comprising:
storing an initial estimate of TES electrode apparatus units associated with a wearable neurostimulator apparatus in the wearable neurostimulator apparatus or a handheld processor in communication with the wearable neurostimulator apparatus;
detecting use of a TES electrode apparatus unit and updating the initial estimate to a current estimate of TES electrode apparatus units associated with the wearable neurostimulator apparatus, in the wearable neurostimulator apparatus or a handheld processor in communication with the wearable neurostimulator apparatus;
comparing the current estimate to a threshold value and shipping, delivering, or otherwise supplying one or more new TES electrode apparatus units to the user if the current estimate falls below the threshold value; and
updating the current estimate to include the one or more new TES electrode apparatus units.

2. The method of claim 1, wherein the current estimate comprises a fractional number to reflect a partially used TES electrode apparatus unit.

3. The method of claim 1, wherein detecting use of the TES electrode apparatus unit comprises detecting use by the wearable neurostimulator apparatus when the TES electrode apparatus unit is coupled to the wearable neurostimulator apparatus.

4. The method of claim 1, wherein detecting use comprises identifying a specific TES electrode apparatus unit by one or more of RFID, NFC, optical code sensing, QR code detection, identification chip detection.

5. The method of claim 4, wherein detecting use of the specific TES electrode apparatus unit comprises determining a remaining functional lifetime of the TES electrode apparatus unit based on a comparison of a manufacturing specification and a stimulation parameter.

6. The method of claim 5, wherein the stimulation parameter is total net charge transfer.

7. The method of claim 5, wherein the stimulation parameter is the duration of electrical stimulation.

8. The method of claim 1, wherein detecting use comprises indirectly detecting use based on activity of the wearable neurostimulator apparatus, wherein the TES electrode apparatus unit is coupled to the wearable neurostimulator apparatus.

9. The method of claim 8, wherein detecting use comprises correlating a physiological measurement with activity of the wearable neurostimulator apparatus.

10. The method of claim 8, wherein the detecting use comprises measuring activity of the wearable TES electrode using an accelerometer and/or gyroscope associated with the wearable neurostimulator apparatus.

11. The method of claim 8, wherein the detecting use comprises an analyzing online activity of the user.

12. The method of claim 8, wherein the detecting use comprises analyzing a semantic or other text or voice analysis of the user.

13. The method of claim 1, further comprising storing the current estimate of TES electrode apparatus units in a machine-readable memory of the wearable neurostimulator apparatus.

14. The method of claim 1, further comprising storing the current estimate of TES electrode apparatus units in a smartphone or other personal computing device.

15. The method of claim 1, wherein the estimate of TES electrode apparatus units remaining for the user is transmitted via the Internet and stored in a remote server.

16. The method of claim 1, further comprising setting, by the user, the threshold value.

17. The method of claim 1, further comprising determining the threshold value for sending one or more new electrode apparatus units based on a pattern of the user's use of the wearable neurostimulator apparatus.

18. The method of claim 17, further comprising periodically updating the threshold value based on the user's pattern of use of the wearable neurostimulator apparatus.

19. The method of claim 1, further comprising allowing the user to select a number of TES electrode apparatus units supplied upon reaching the threshold value.

20. The method of claim 1, further comprising automatically selecting a number of electrode apparatus units supplied to a user upon reaching the threshold value is determined based on a pattern of use of the wearable neurostimulator apparatus.

21. The method of claim 20, wherein the number of electrode apparatus units supplied to a user upon reaching the threshold value is updated periodically based on a pattern of use or delay in use of the wearable neurostimulator apparatus.

22. The method of claim 1, wherein shipping, delivering, or otherwise supplying one or more new TES electrode apparatus units to the user is achieved by a local courier or delivery service.

23. The method of claim 1, wherein the shipping, delivering, or otherwise supplying one or more new TES electrode apparatus units to the user is achieved by fulfillment from a regional or national warehouse.

24. The method of claim 1, wherein the speed of shipping, delivering, or otherwise supplying one or more new electrode apparatus units to the user is performed based on a pattern of use of the wearable neurostimulator apparatus by the user.

25. The method of claim 1, wherein updating the current estimate occurs at a fixed time after shipping based on the shipment method and speed.

26. The method of claim 1, wherein updating the current estimate occurs once a tracking, delivery signature, or other delivery notice is received.

27. The method of claim 1, wherein updating the current estimate occurs when a first electrode apparatus of the shipment is detected by the wearable neurostimulator apparatus or a handheld processor in communication with the wearable neurostimulator apparatus.

28. The method of claim 1, further comprising selecting from a plurality of choices for shipping.
